(19) United States
(12) Patent Application Publication
(10) Pub. No.: US 2009/0157098 A1
(43) Pub. Date: Jun. 18, 2009

(54) SURGICAL WIRE CLOSURE DEVICES

(75) Inventor: Farid A. Meybodi, Shell Cove (AU)

Correspondence Address: NEXUS LAW GROUP LLP, 1500 - 701 WEST GEORGIA STREET, VANCOUVER, BC, V7Y 1C6, CA

(73) Assignee: MD TECHNOLOGY CORP., North Vancouver (CA)

(21) Appl. No.: 11/954,611

(22) Filed: Dec. 12, 2007

(51) Int. Cl. A61B 17/10

(52) U.S. Cl. 606/139

(57) ABSTRACT

Surgical wire closure devices, which in some embodiments may be incorporated onto other surgical devices such as surgical pliers, comprising an elongate arm having a hook at one end and a shaft at the other end, wherein the axis of the arm is angled in relation to the axis of the shaft, and wherein the shaft is rotatably connected to a handle. The hook is inserted under a first node of surgical wire during approximation, followed by rotation of the hook about the axis of the shaft, which is accomplished by a slight pivoting motion of the surgeon's hand about the wrist as he or she grasps the handle.

### BACKGROUND OF THE INVENTION

The present invention relates generally to surgical tools, and more particularly to surgical tools for joining two ends of surgical wires together during the procedure of approximation of two bony segments or other hard tissues.

Every year there are thousands of surgeries in North America, including cardiothoracic, neurosurgical and orthopedic operations. It is advantageous in all operative procedures to shorten the operation time. The wound closure steps, which include closing and suturing, have been considered as one of the more sensitive skills in the various types of surgeries, and represent the period of time during surgeries that is most amenable to new time saving techniques or instruments.

Many surgeries require making an incision into hard tissue and bones, such as the sternum of the chest wall, in order to access the targeted internal organ. Similarly, many surgeries such as open cardiac, orthopedic, maxillofacial and neurosurgery involve hard tissues like bone, fascia and tendons. Closure of these hard tissues consumes considerable time and effort, whereas it is advantageous to the health and safety of the patient that operative procedures be fast and precise. Accordingly, it would be advantageous to have novel apparatus and methods of efficient wound closure as a means of shortening the duration of operative procedure.

Stainless steel wires are widely used to approximate two bony segments or other hard tissues that require more force to be closed. For example, in cardiothoracic (open cardiac) surgeries, a mid sternotomy is needed for access to the heart and great vessels. To close the osteotomies, stainless steel wires are typically inserted into the area that has been prepared. The incision is closed by twisting the two heads of stainless steel wires in both sides of two halves of bone and in different locations. To achieve this, most of surgeons use the surgical pliers, and by several rotations of the instrument, they close two halves of the bone together. The time required to close each wire is an important factor in the procedure, especially for high risk and critically ill patients such as cardiac patients. Stainless steel wires are not easy to work with since they can be broken or loosened due to sudden change of pressure during the twisting or the traction of two heads of wires to fasten and close the incisions. Accordingly, it would be advantageous to have an apparatus for efficiently twisting the two ends of stainless steel wire during surgical procedures without damaging the wires.

### SUMMARY OF THE INVENTION

The present invention provides novel surgical wire closure devices, which in some embodiments may be incorporated onto surgical pliers, that can pull and twist the two heads of the suture wires efficiently and precisely without causing damage to the wires. The devices of the present invention comprise an elongate arm having a hook at one end and a shaft at the other end, wherein the axis of the arm is angled in relation to the axis of the shaft, and wherein the shaft is rotatably connected to a handle. The hook is inserted under a first node of surgical wire during approximation, followed by rotation of the hook about the axis of the shaft, which is accomplished by a slight pivoting motion of the surgeon's hand about the wrist as he or she grasps the handle. The rotations of the hook create a fine and regular helix in the wires and fasten them in a clean and precise manner. The even pressure of the hook's rotation prevents damage to the wires and inhibits breaking of the wires. The present invention facilitates performing wound closure or approximations in less time and in more comfort because of relatively small rotational movement required of the surgeon's hand, as compared to the forearm twisting motions required when using conventional wire closure devices and techniques.

### BRIEF DESCRIPTION OF THE DRAWINGS

For a better understanding of the present invention and to show more clearly how it may be carried into effect, reference will now be made by way of example to the accompanying drawings.

FIG. 1 is a top plan view of an embodiment of the present invention; and

FIG. 2 is a top view of the device in FIG. 1 with a top section of the enlarged handle portion removed to reveal the rotatable attachment of the wire twister to the handle.

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention. Therefore, specific details disclosed herein are not to be interpreted as limiting, but rather as a basis for the claims and as a representative basis for teaching one skilled in the art to employ the present invention.

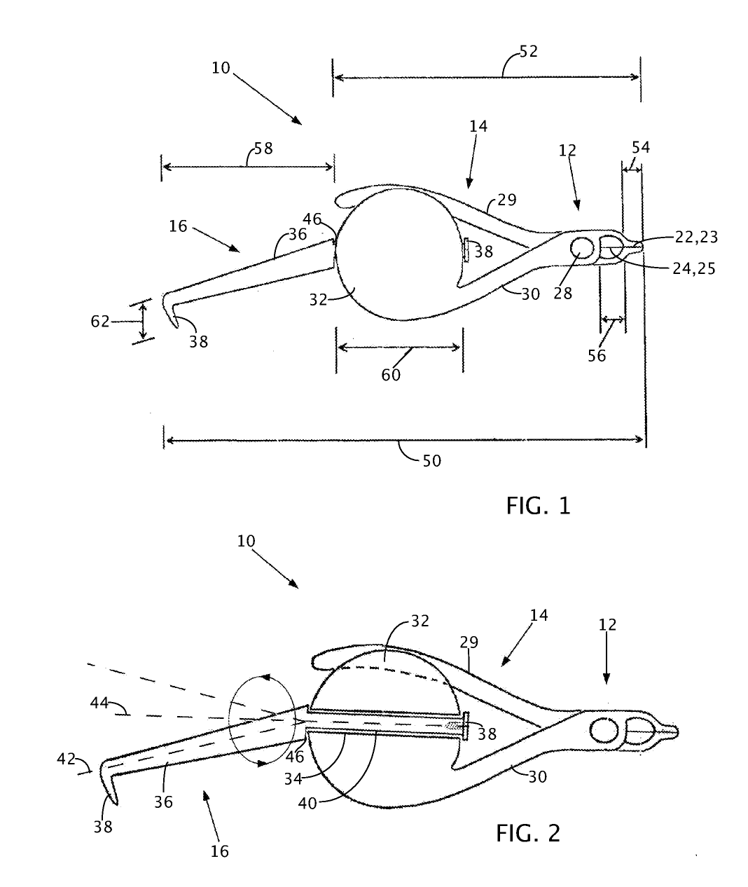

Referring to FIG. 1, an embodiment of a surgical wire closure device of the present invention is generally indicated at 10. Device 10 comprises a pliers portion 12 having an ergonomic handle 14 that is coupled to a wire twister 16 for twisting of the two heads of metal suture wires together during surgical procedures. The pliers 12 may be a design suitable for use in surgery and would be apparent to persons skilled in the art. In the illustrated embodiment, the pliers 12 generally comprises a pair of jaws 20,21 each having complimentary gripping portions 22,23 and wire cutting portions 24,25. The jaws are hingedly connected to each other by joint 28 as is known in the art. A handle 29 or 30 is attached to its respective jaw for grasping and manipulating the pliers. Handle 30 terminates in an enlarged bulbous portion 32 that is designed and proportioned to fit ergonomically within the palm of the user.

Referring to FIG. 2, a longitudinal bore 34 is provided through the bulbous portion 32 along the central axis of the device. Wire twister 16 comprises an elongate member or arm 36 having a hook 38 at one end and a shaft 40 at the other end. The axis 42 of the elongate arm is offset from the axis 44 of the shaft by between 10 and 30 degrees, and preferably by 15 degrees. The shaft 40 has a smaller cross-sectional diameter than the arm, thereby defining a shoulder 46 at the juncture of the arm and the shaft, and is dimensioned to fit closely but rotatably within bore 34 of the bulbous portion 32. The shaft is provided with a stop screw 38 that is larger than the bore 32, and together with the shoulder 46 that is also larger than the bore, retains the shaft within the bore. Accordingly, the wire twister 16 is able to rotate relative to the rest of the device. Alternatively, the shaft 40 of the wire twister 16 may be journalled for rotation within the bulbous portion by bushings or bearings, and such embodiments are within the scope of the present invention. Removal and replacement of the wire twister may be accomplished by removing stop screw 38, which enables the use of different sized shafts and hooks for different applications.

Referring again to FIG. 1, in a preferred embodiment of the present invention, the overall length 50 of the device is 28.5 cm; the length 52 of the pliers is 17.5 cm; the length 54 of the gripping portions is 1.2 cm; the length 56 of the wire cutting portions is 1.6 cm; the length 58 of the wire twister is 11 cm; the length 60 of the bulbous portion is 5.5 cm; and the length 62 of the hook is 1 cm.

The present invention may be made of non-magnetic and non-corrosive stainless steel, or of other suitable material approved for use in surgical devices. The illustrated embodiment is designed for holding and traction of the two heads of suture wire. The offset angle of arm relative to the shaft transmits a relatively minor wrist pivoting movement of the surgeon's hand into rotation of the wire twister such that the hook is able to twist the two heads of wire together. This process is fast and precise, and reduces the possibility of damaging or breaking of the suture wires.

In use of the device, an initial primary traction of the two heads of the wires from the two halves of an incision area (for example, sternum or bone) is performed and the pliers are used to complete the first surgical node by conventional rotation of the pliers. Then the tip of the hook is inserted under the first node, followed by rotation of the hook about the axis 44 of the shaft, which is accomplished by a slight pivoting motion of the surgeon's hand about the wrist. These rotations of the hook create a fine and regular helix in the wires around the vertical axis and fasten them in a clean and precise manner. The even pressure of the hook's rotation prevents damage to the wires and inhibits breaking of the wires. Breakage of a wire during closure could result in the surgeon having to remove the wire and replace it with a new one, thereby complicating and lengthening the procedure. The present invention facilitates performing wound closure in less time and more comfort because of relatively small rotational movement required of surgeon's hand as compared to the forearm twisting motions required when using conventional wire closure devices and techniques. Once the surgeon is satisfied with the number of nodes in the twisted wires, the extra portion of the suture wires can be cut with the wire cutting portion of the device.

It has been found that the illustrated embodiment of the present invention can reduce the time period of wound closure to one-third or one-fourth of the time it takes with conventional devices and techniques, depending on wound location. In addition, the device can fasten suture wires in recessed cavities or deep (and otherwise inaccessible) areas, which is an important efficiency in procedures such as maxillofacial surgeries.

In alternative embodiments, the present invention may comprise a handle and a wire twister portion, without the pliers portion. In further alternative embodiments of the present invention, the wire twister and handle may be incorporated together with another surgical instrument.

While the above description and illustrations constitute preferred or alternate embodiments of the present invention, it will be appreciated that numerous variations may be made without departing from the scope of the invention.

The embodiments of the invention in which an exclusive property or privilege is claimed are defined as follows:

1. A surgical wire closure device comprising:
a handle; and
an elongate member having a hook portion at one end and a shaft at the other end, wherein the axis of the elongate member is angled in relation to the axis of the shaft, and wherein the shaft is rotatably connected to the handle.

2. The device as claimed in claim 1, wherein the axis of the elongate member is at an angle in the range of 10 to 30 degrees to the axis of the shaft.

3. The device as claimed in claim 1, wherein the axis of the elongate member is at an angle of 15 degrees to the axis of the shaft.

4. The device as claimed in claim 1, further comprising surgical pliers connected to the handle.

5. The device as claimed in claim 4, wherein the axis of the elongate member is at an angle in the range of 10 to 30 degrees to the axis of the shaft.

6. The device as claimed in claim 4, wherein the axis of the elongate member is at an angle of 15 degrees to the axis of the shaft.
